(19) United States
(12) Patent Application Publication
(10) Pub. No.: US 2026/0146998 A1
(43) Pub. Date: May 28, 2026

(54) Bio-Ink and Its Preparation and Application

(71) Applicant: Xudong Cao, Ottawa (CA)

(72) Inventors: Xingkai Hao, Hangzhou (CN); Man Lu, Ontario (CA); Xudong Cao, Ottawa (CA)

(21) Appl. No.: 18/961,201

(22) Filed: Nov. 26, 2024

(51) Int. Cl. G01N 33/569; C09D 11/03; C09D 11/102; C12N 15/115; G01N 33/543

(52) U.S. Cl. CPC G01N 33/56983; C09D 11/03; C09D 11/102; C12N 15/115; G01N 33/54388; C12N 2310/16; G01N 2333/185

(57) ABSTRACT

The present invention discloses a bio-ink and its preparation procedures, including a) coupling dendritic polymers with capturing ligands to form polymer-ligand conjugate complexes, and b) coupling the resulting polymer-ligand conjugate complexes with specific proteins to obtain the bio-ink. The bio-ink disclosed in this invention can have many different applications in biosensing and detection and can be conjugated with different capturing ligands and proteins according to different detection requirements. It can also be applied to various detection platforms. As one example shown in the invention disclosure, the bio-ink is used for dengue virus detection. The use of the bio-ink has been shown to significantly improve the target capturing ability on the T-line, and as a result improves the sensitivity for dengue virus detection.

### CROSS-REFERENCE TO RELATED APPLICATIONS

This application contains a sequence listing submitted as a XML file, named “29994HIO-US-AMD-Sequence-Listing.xml” and created on Feb. 24, 2025, with 4.40 kilobytes in size. The material in the above-identified XML file is incorporated herein by reference in its entirety.

### TECHNICAL FIELD

The present invention belongs to the field of biotechnology detection technology, specifically related to a bio-ink, its preparation method and its applications.

### BACKGROUND

Lateral Flow Assay (LFA) is a rapid detection platform technology that is easy to operate, low-cost, and capable of producing results within 30 minutes. Due to its quick sample-to-result turnaround, LFA has been widely used in many applications, including but not limited to COVID-19 infection detection and pregnancy tests. Despite its excellent performance in detection speed and specificity, there is still room for improvement in its sensitivity. To enhance the sensitivity of this LFA platform technology, many strategies have been developed, including employing novel nanoparticles such as silver nanoparticles and quantum dots to replace traditional gold nanoparticles to enhance the intensity of colorimetric signals. However, even with these signal intensification strategies, the sensitivity remains insufficient for many applications that require low detection limits.

Most LFA test strips use a sandwich detection format. In a positive test, the targeted analytes in the sample bind to the primary ligands (e.g., primary antibodies or primary aptamers) labeled with gold nanoparticles (AuNPs) to form conjugate complexes that move along the test strip via capillary actions. The complexes are then specifically captured and enriched on the test line (T-line) because of their specific interactions with the secondary ligands (e.g., secondary antibody or secondary aptamer) with which the T-line is printed, producing a visible signal through the aggregation of the AuNPs, the signaling agents. One of the key reasons for the low LFA detection sensitivity is due to the sub-optimal capturing efficiency of the secondary ligands printed on the T-line to capture the signaling agent complexes; this loss of signals on the T-line is particularly pronounced in samples where targeted analyte concentrations are already low, leading to low detection sensitivities.

### SUMMARY OF THE INVENTION

The objective of the present invention is to solve the low detection sensitivity issue that faces the LFA detection platform by proposing a bio-ink as the solution and disclosing its preparation method.

To realize the above objectives, the invention employs the following technical scheme:

A method to prepare a bio-ink, including: a) coupling a dendritic polymer with capturing ligands to form a multi-handled polymer-ligand conjugate complexes; b) coupling the multi-handled polymer-ligand conjugate complexes obtain in the previous step with a specific protein to obtain the bio-ink.

Dendritic polymers are spherical polymers with multiple functional groups, such as —OH, —COOH, and —NH₂ on the polymer surfaces. Due to their unique structures, these dendritic polymers can be used as perfect templates to conjugate multiple copies of capturing ligands (such as aptamers) to prepare multi-handled polymer-ligand conjugate complexes. When used in biosensors, these multi-handled polymer-ligand conjugate complexes can significantly enhance the probabilities of target-ligand interactions and capturing efficiencies, thereby improving detection performances. Furthermore, coupling specific proteins with the polymer-ligand conjugate complexes ensures that the final bio-ink can be securely anchored in the T-line position—where the bio-ink is printed—in the LFA strip. Finally, due to the unique anti-fouling properties of the dendritic polymer[1, 2], the application of dendritic polymers in the bio-ink significantly reduces the interaction of the bio-ink with other molecules at the T-line position, thereby effectively reducing the probability of false positive.

Various types of dendritic polymers, capturing ligands, and specific proteins can be selected for the present invention, wherein the dendritic polymer is either a poly(amidoamine) (PAMAM) dendrimer, poly(propylenimine), triazine, phosphorus, or polyether dendritic polymer. Preferably, the dendritic polymer is a PAMAM dendrimer. The PAMAM dendrimer can be one or combinations of the dendrimers from generation 2 to generation 9, and preferably, the PAMAM dendrimer includes generation 6.5 PAMAM dendrimer.

Preferably, the capturing ligand is either an aptamer, antibody, peptide or molecularly imprinted polymer. The sequence of the aptamer is preferably as shown in SEQ ID NO. 1.

Preferably the specific protein includes streptavidin, avidin, and bovine serum albumin.

The present invention also describes a bio-ink prepared by the above mentioned preparation method.

Furthermore, the present invention describes the application of the bio-ink in test strip kits.

The bio-ink prepared in the present invention can have various applications, such as in lateral flow assays.

The present invention also describes a dengue virus test kit using a lateral flow test strip format. The lateral flow test strip includes a sample pad, a conjugate pad, and a membrane with a T-line and a control line (C-line), characterized in that the T-line contains bio-ink for detecting dengue virus. The preparation process of the bio-ink includes: a) coupling PAMAM dendrimers with capturing ligands to prepare multi-handled conjugate complexes, wherein the capturing ligands are amine-functionalized aptamers with the sequence as shown in SEQ ID NO. 1, and b) coupling the multi-handled conjugate complexes with streptavidin to obtain the bio-ink.

In the present invention, the bio-ink at the T-line combines streptavidin, dendritic polymer (such as generation 6.5 PAMAM dendrimer (G6.5)), and secondary aptamer (sAptamer) to prepare the bio-ink. The specific preparation steps of the bio-ink are as follows: 1) by coupling PAMAM dendrimer G6.5 with sAptamer, a G6.5-sAptamer complex is prepared. This resulting complex features multiple sAptamers on the multi-armed dendritic polymer template, significantly increasing the number of capturing ligands and their target-binding abilities, thereby increasing the opportunities to capture target molecules. 2) the synthesized G6.5-sAptamer complex is further reacted with streptavidin to allow streptavidin to be attached to the G6.5-sAptamer complex to obtain the bio-ink. Streptavidin is used as an anchor molecule via which the bio-ink is physically adsorbed onto the nitrocellulose membrane of rapid test strips. As a result, the resulting bio-ink can be securely anchored at the T-line position where it is printed on the LFA strip.

To enhance the capturing efficiencies of the signaling molecules on the T-line, the present invention tries to increase the density of the capturing ligands on the T-line for the signaling molecules and to enhance the overall binding affinities. The bio-ink of the present invention uses the streptavidin-dendrimer-aptamer conjugates at the T-line to replace the traditional secondary capturing ligands (e.g., aptamers or antibodies) on the T-line of test strips. This bio-ink significantly improves the target capturing efficiencies at the T-line, thereby enhancing detection sensitivities.

Other components of the lateral flow test strip in the present invention can be prepared using conventional methods. Preferably, the conjugate pad is embedded with AuNPs-labeled oligonucleotides, with the sequence of the oligonucleotide being shown in either SEQ ID NO. 2 or SEQ ID NO. 3; the C-line contains streptavidin-biotin-oligonucleotide complexes, with the sequence of biotin-oligonucleotide being shown in SEQ ID NO. 4.

The benefits of the invention compared with prior arts are:

(1) The bio-ink in the present invention allows multiple copies of capturing ligands to be conjugated to multi-handled dendritic polymer templates, significantly increasing the number of capturing ligands available for signaling molecule recognition and capturing on the T-line, thereby increasing their target capturing efficiencies. Subsequent conjugation of the polymer-ligand conjugate complexes with proteins, to result in the bio-ink, ensures that the bio-ink can be securely anchored to the T-line where it is printed.

(2) The bio-ink in the present invention can be used to conjugate with different capturing ligands and proteins according to different detection applications and various detection platform requirements. For example, when the bio-ink is printed on the T-line of a test strip, it can increase the capturing ligand density on the T-line, thereby improving target capturing efficiency and enhancing detection sensitivity.

(3) The present invention fully utilizes the unique anti-fouling properties of dendritic polymers to significantly reduce nonspecific interactions between bio-ink and other molecules at the T-line, thereby effectively reducing the probability of false positive.

(4) The bio-ink in the present invention can be used for dengue virus detection by markedly improving the target capturing abilities of the T-line and significantly enhancing the detection sensitivities for the dengue virus in a paper strip test format.

### BRIEF DESCRIPTION OF THE DRAWINGS

In order to better illustrate the technical solutions of the embodiments of the present invention, the descriptions of drawings to illustrate listed embodiments are briefly listed. It should be clear that the drawings in the following description are some embodiments of the present invention, and it is obvious for those skilled in the art to obtain other drawings based on these drawings without creative efforts.

FIG. 1: Overall structure of the bio-ink synthesized using poly(amidoamine) dendrimer generation 6.5 (PAMAM dendrimer G6.5), secondary aptamer (sAptamer), and streptavidin in Embodiment 1 of the present invention;

FIG. 2: Schematic diagram of the overall structure of the lateral flow assay test strip (made using the bio-ink from Embodiment 1) in Embodiment 2 of the present invention;

FIG. 3: Particle size analysis of G6.5, G6.5-sAptamer, and streptavidin-G6.5-sAptamer using dynamic light scattering;

FIG. 4: Number of sAptamers conjugated to the G6.5 multi-handled template;

FIG. 5: Characterization of AuNPs and AuNPs-pAptamer conjugates (where A and B are TEM images of AuNPs and AuNPs-pAptamer conjugates; C and D are dynamic light scattering and UV-Vis analysis of the AuNPs and AuNPs-pAptamer conjugates).

FIG. 6: Different concentrations of streptavidin-dendrimer-sAptamer complexes used on the T-line and its effects on false positive test results (where A and B are test strips prepared using different concentrations of streptavidin-G6.5-sAptamer and streptavidin-G3.5-sAptamer conjugates; the X-axis represents streptavidin-dendrimer-sAptamer concentrations used to print the T-lines; the Y-axis represents signal strength measured on the T-line when a blank sample is tested; and the dashed line represents background noise);

FIG. 7: Detection of dengue virus S1 E protein samples (where A shows detection performances of different T-line test strips at different target concentrations; B shows dual T-line testing for streptavidin-sAptamer and streptavidin-G6.5-sAptamer test strips; note that each type of test strip had two T-lines printed in the test area);

FIG. 8: Matrix effect and specificity tests (where A shows detection performance in PBS (pH 7.4) and BSA (1% w/w) solutions; B shows specificity detection of streptavidin-G6.5-sAptamer test strip; target protein is dengue virus S1 E protein, non-target proteins include dengue virus S2, S3, and S4 E proteins and Zika virus (ZIKV) E protein).

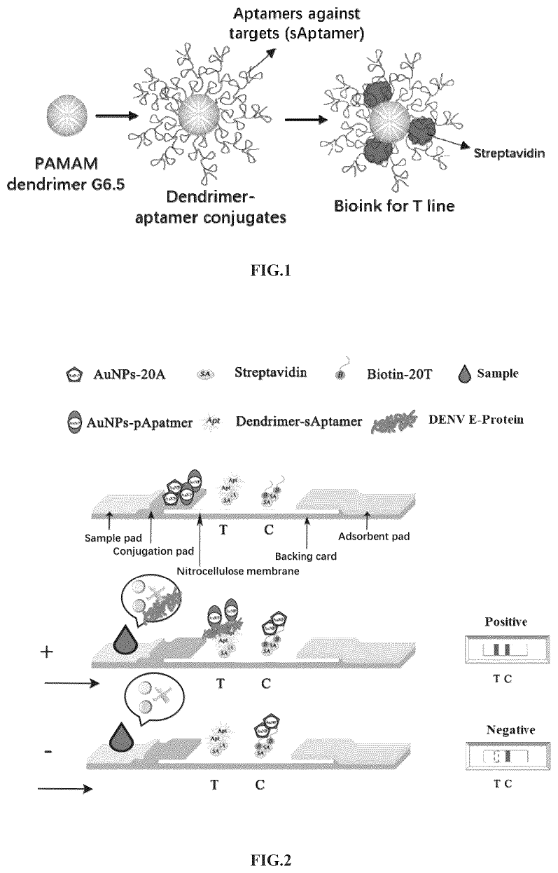

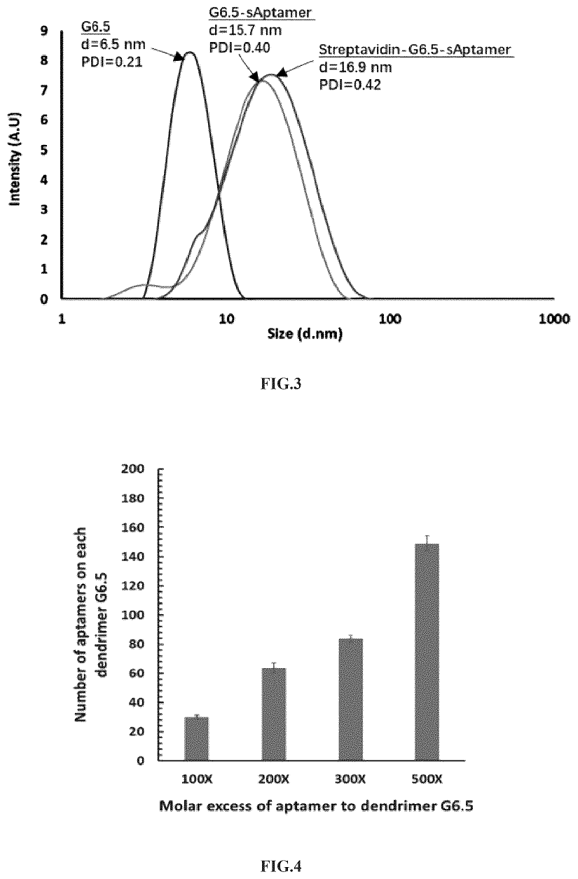

### DETAILED DESCRIPTION

The technical solutions in the embodiments of the present invention are clearly described below with reference to the drawings in the embodiments of the present invention. It should be noted that the described embodiments are some, not all, embodiments of the present invention. All other embodiments that can be derived by a person skilled in the art from the embodiments given herein without making any creative effort shall fall within the protection scope of the present invention.

It is understood that the terminology used in the description of the invention herein is for the purpose of describing particular embodiments only and is not intended to be limiting of the invention. As used in the specification of the present invention and the appended claims, the singular forms “a,” “an,” and “the” are intended to include the plural forms as well, unless the context clearly indicates otherwise.

It is also to be understood that the term “and/or” as used in this specification and the appended claims refers to and includes any and all possible combinations of one or more of the associated listed items.

### Embodiment 1

### Preparation of the G6.5-Aptamer-Streptavidin Based Bio-Ink, Detailed Steps as Follows:

### 1. Activation of PAMAM Dendrimer Generation 6.5 (G6.5)

To activate G6.5 molecules, 10 μL of 100 μM carboxyl-functionalized G6.5 was added to 500 μL of an activation solution containing 0.1 M NHS, 0.1 M EDC, and 0.1 M MES (pH 6.0), and reacted at room temperature for 15 minutes to convert the carboxyl groups on the surfaces of the G6.5 molecules to NHS-ester groups. Subsequently, the resulting solution was transferred to a 30 KD Amicon® Ultra centrifugal filter unit (Millipore Sigma, Germany) and centrifuged at 8000×g for 10 minutes to remove unreacted reagents, and the activated G6.5 molecules were collected.

2. Conjugation of sAptamer with G6.5

To conjugate sAptamers (secondary aptamer) with G6.5 molecules, 52 μL of 1 mM amino-modified sAptamer (NH2-sAptamer) was added to the above activated G6.5 molecules and was reacted at room temperature for 15 minutes to obtain G6.5-sAptamer complexes. The sequence of the sAptamer (i.e., capturing ligand) is shown in SEQ ID NO. 1: 5′-/NH2-C6/ATC CGT CAC ACC TGC TCT AGG CTG TGG TGA CGT ACC AGG GGA GTG GGT CGC CAG TGG TGT TGG CTC CCG TAT-3′.

3. Conjugation of Streptavidin with G6.5-sAptamer Complexes

To conjugate streptavidin with the G6.5-sAptamer complexes obtained in the step above, 40 μL of 1 mM streptavidin was added to the G6.5-sAptamer complex mixture, and the reaction mixture was reacted at room temperature for 30 minutes. Finally, the resulting solution was transferred to a 100 KD Amicon® Ultra centrifugal filter unit and centrifuged at 8000×g for 10 minutes to remove unreacted reactants to result in purified streptavidin-G6.5-sAptamer conjugates.

4. Characterization of Streptavidin-G6.5-sAptamer

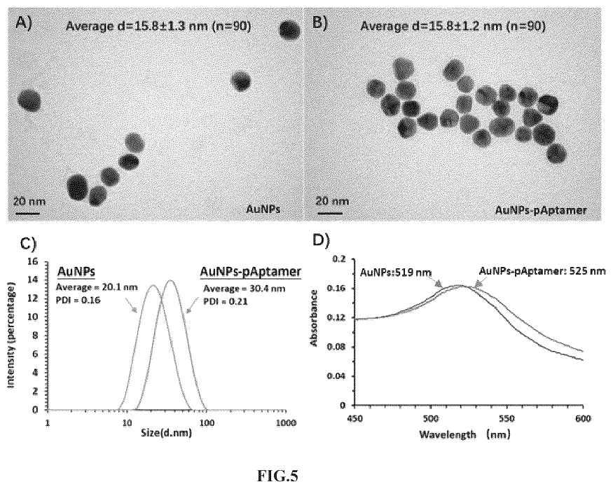

To confirm the successfulsynthesis of streptavidin-G6.5-sAptamer, dynamic light scattering (DLS) was used to follow the particle sizes of G6.5, G6.5-sAptamer, and the streptavidin-G6.5-sAptamer complexes in the preparation process, respectively. As shown in FIG. 3, in comparison with the size of G6.5, the size of the G6.5-sAptamer complex significantly increased (6.5 vs. 15.7 nm), suggesting that the sAptamer was successfully attached to the G6.5 molecules. In addition, when streptavidin was subsequently conjugated to the G6.5-sAptamer complex to form streptavidin-G6.5-sAptamer, the particle size further increased to 16.9 nm, indicating that the streptavidin-G6.5-sAptamer complex was successfully synthesized.

In addition, to study the number of sAptamer molecules conjugated to each multi-handled G6.5 molecule, the present invention used FAM labeled fluorescent secondary aptamer (sAptamer-FAM) to prepare the G6.5-sAptamer complexes in order to calculate the number of aptamer molecules on each G6.5 molecule. As shown in FIG. 4, with increasing sAptamer vs. G6.5 ratios in the initial reaction feed, the number of sAptamer molecules conjugated to each G6.5 molecule increased. When the initial feed ratio of sAptamer vs. G6.5 was 500 (the highest concentration achievable due to raw material concentration limitations), the number of sAptamer molecules conjugated to each G6.5 molecule reached a maximum value at 150 (i.e., 150 sAptamer molecules per G6.5 molecule).

### Embodiment 2

### Preparation of the Dengue Detection Kit

### 1. Synthesis of Oligonucleotide Labeled AuNPs (AuNPs-20A and AuNPs-pAptamer) for Conjugate Pads

To synthesize oligonucleotide labeled AuNPs for conjugate pads, AuNPs were prepared first. Specifically, a 50 mL of 0.01% (w/v) HAuCl₄ solution was heated and stirred at 200 rpm until boiling. Subsequently, 1 mL of 1% (w/v) trisodium citrate solution was added to the boiling solution and heated for an additional 2 minutes with continuous stirring until the final color of the reaction mixture resembled that of red wine. The resulting AuNPs solution was cooled to room temperature and stored at 4° C.

To synthesize oligonucleotide labeled AuNPs, 6 μL of 1 mM disulfide functionalized oligonucleotides—either primary aptamer (pAptamer) or a stretch of 20A—were treated with 122.4 mM freshly prepared tris(2-carboxyethyl)phosphine hydrochloride (TCEP-HCl) at room temperature for 2 hours to obtain —SH functionalized oligonucleotides. The resulting —SH functionalized oligonucleotides were added to a conjugation solution containing 2.9 mL AuNPs and 30 μL of Na₂HPO₄ solution (1 M, containing 1% w/w sodium dodecyl sulfate (SDS)). The reaction mixture was left in a dark environment at room temperature for 1 hour. Subsequently, a salt aging solution (containing 2 M NaCl, 0.01 M Na₂HPO₄, and 0.01% (w/w) SDS) was gradually added to the reaction solution to increase the NaCl concentration to 0.1 M, followed by 10 seconds of sonication and subsequent incubation at room temperature for 20 minutes. This salt aging process was repeated 7 times, and each time the NaCl concentration used in the process was increased by 0.1 M increment until a final concentration of 0.7 M NaCl was reached. The resulting reaction mixture was then left at room temperature for 12 hours. Finally, the solution was centrifuged at 13500×g for 20 minutes to remove the supernatant, and the precipitate (i.e., oligonucleotide labeled AuNPs) was collected and resuspended in 0.01 M PBS (pH 8.2) containing 0.5% PEG 2000, 5% sucrose, 0.25% Tween 20, and 1% BSA.

The sequence of pAptamer is shown as in SEQ ID NO.2: 5′-/SH-C6/ATC CGT CAC ACC TGC TCT GGT AGT TCG TTC TCT TGT ACA CTC TGG AAT TTA GGG TGG TGT TGG CTC CCG TAT-3′; and the sequence of 20A is shown as in SEQ ID NO.3: 5′-/SH-C6/AAAAAAAAAAAAAAAAAAAA-3′.

### 2. Synthesis of Streptavidin-20T Conjugates for C-Line

To synthesize streptavidin-20T conjugates, 4 μL of 100 μM Biotin-20T was reacted with 50 μL of 0.125 mg/mL streptavidin and incubated at room temperature for 30 minutes to obtain streptavidin-20T conjugates. The sequence of Biotin-20T is shown as in SEQ ID NO.4: 5′-/Biotin-C6/TTTTTTTTTTTTTTTTTTTT-3′.

### 3. Printing of T-line and C-line

To print T-line and C-line on the paper strip, the streptavidin-G6.5-sAptamer (i.e., the bio-ink) and streptavidin-20T conjugates were used to be individually dispensed on the nitrocellulose membranes of the paper strip using a Biodot 3050 dispensing platform (Irvine, CA). Subsequently, the nitrocellulose membrane was dried at 37° C. for 1 hour.

### 4. Assembly of Test Strips

To assemble the test strips, conjugate pads that were pre-configured to contain both AuNPs-20A and AuNPs-pAptamer were placed on top of the nitrocellulose membrane. Subsequently, the sample pads were placed over the conjugate pads by ensuring precise alignments between the two layers of the membranes. The assembled test strips were then placed on a plastic backing to ensure stability during applications. Finally, the test strips were cut to a standard width of 3 mm and stored in a desiccator before use.

### Characterizations:

1. Characterization of AuNPs and AuNPs-pAptamer conjugates

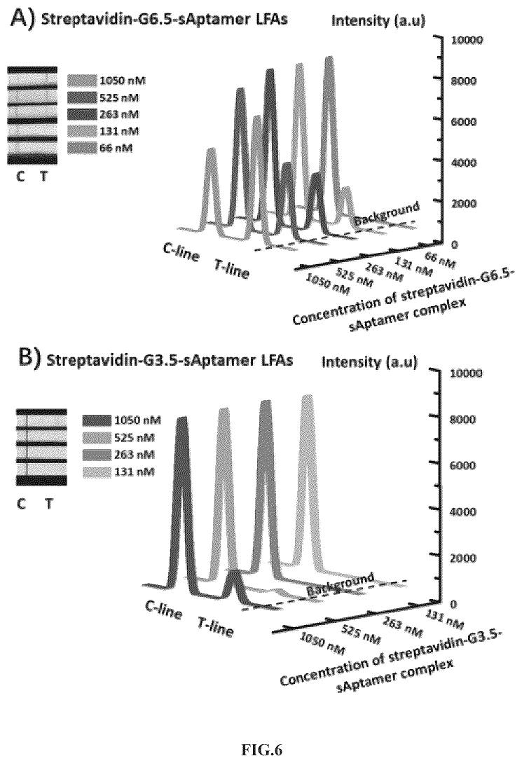

To confirm the successful synthesis of AuNPs and AuNPs-pAptamer, transmission electron microscopy (TEM), dynamic light scattering (DLS), and UV-Vis spectroscopy were used. As shown in FIG. 5A, the TEM image demonstrated that the synthesized AuNPs appeared to assume uniform spherical shapes with an average diameter of 15.8 nm±1.3 nm (n=90). The TEM image of the AuNPs-pAptamer (shown in FIG. 5B) showed no significant difference in particle sizes in comparison with the AuNPs, with an average diameter of 15.8±1.2 nm (n=90). Moreover, DLS results (FIG. 5C) indicated that the average size of AuNPs-pAptamer was larger than that of AuNPs (30.4 nm vs. 20.1 nm), suggesting the successful conjugation of aptamers onto AuNPs. Additionally, UV-Vis spectra (FIG. 5D) showed a 6 nm red shift in the maximum absorption peak of AuNPs-pAptamer compared to AuNPs (525 vs. 519 nm), confirming the successful conjugation of AuNPs with pAptamers.

### 2. Test Strip Performances

The concentration of detection ligands printed on the T-line is a critical factor affecting detection test strip performances. According to the literature, excessively high concentrations of detection ligands printed on the T-line can potentially lead to false positive results due to physical blocking effects by the T-line while low concentrations result in low detection sensitivities. Therefore, the ideal ligand concentrations used on the T-line should be as high as possible without causing false positives. In this embodiment, the optimal concentrations of the bio-ink used on the T-line were carefully studied.

Specifically, different concentrations of the streptavidin-G6.5-sAptamers were printed on the T-lines of different test strips, and the intensities of color developments of both the C-line and T-line of negative controls (i.e., PBS) were measured and compared. As shown in FIG. 6A, the intensities of the false positive signals on the T-line decreased with decreasing streptavidin-G6.5-sAptamers concentrations used on the T-line. No false positive signal was observed when the streptavidin-G6.5-sAptamers concentration was reduced to 66 nM or below; thus 66 nM was selected as the optimal condition. Notably, at the highest streptavidin-G6.5-sAptamer concentration (i.e., 1050 nM), the C-line signal was significantly lower (4000 vs. 8000 a.u.) compared to other concentrations (p<0.05). This is likely because that the high streptavidin-G6.5-sAptamers concentration used on the T-line blocked the movement of control line signals (i.e., AuNPs-20A) to the C-line, resulting in a much lowered C-line signal intensity.

Similarly, the optimal concentration of streptavidin-G3.5-sAptamers was also investigated. As shown in FIG. 6B, the false positive signal intensities on the T-line showed a decreasing trend similar to that observed in FIG. 6A. No false positive signal was observed when the concentration of the streptavidin-G3.5-sAptamers was at or below 263 nM; thus 263 nM was determined as the optimal concentration for streptavidin-G3.5-sAptamers printed on the T-line. Furthermore, at the optimal concentrations for both G3.5 and G6.5 based test strips, the total amount of the aptamers available on the T-line for target capturing was compared. The results indicated that the number of aptamers on the T-line was similar for both bio-inks, suggesting that both G3.5 and G6.5 based LFAs likely have similar target capturing performances.

3. Detection of Dengue Virus Targets (Target: DENV S1 E Protein) To evaluate the performance of paper strips prepared using streptavidin-G6.5-sAptamers in detecting dengue virus (DENV) S1 E protein samples, a series of experiments were conducted, including using different concentrations of target samples. The results of the streptavidin-G6.5-sAptamers test strips were compared with other types of test strips, such as those with streptavidin-G3.5-sAptamers, streptavidin-sAptamers, and antibodies printed on the T-line.

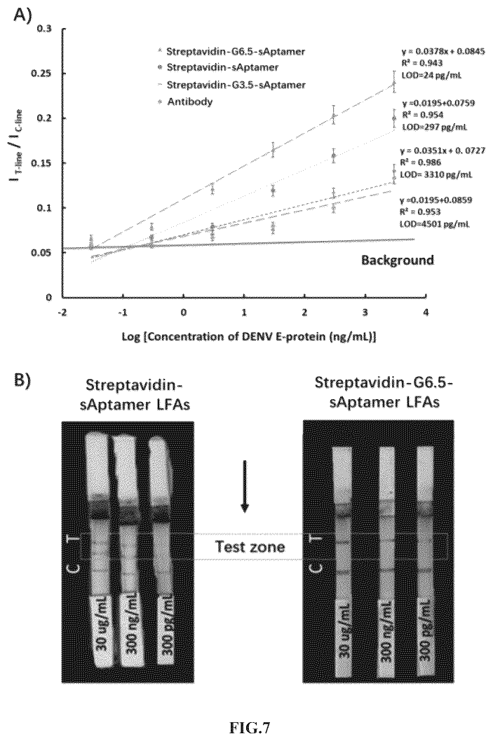

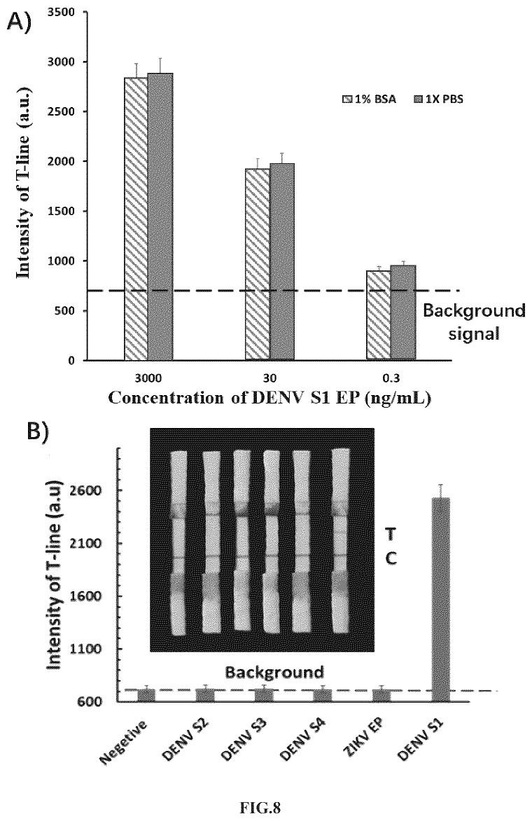

As shown in FIG. 7A, for all concentrations tested, test strips with T-lines printed using streptavidin-G6.5-sAptamers consistently showed higher detection signals on the T-lines in comparison with other types of test strips (i.e., streptavidin-G3.5-sAptamer, streptavidin-sAptamers and antibody-based test strips). Further studies on the detection limits—lowest target concentration where the signal is at least three times the standard deviation of the negative control sample—of all test strips revealed that the streptavidin-G6.5-sAptamers test strips had a remarkably low detection limit of 24 pg/mL, which is approximately 12 times and 138 times lower than the detection limits of the streptavidin-sAptamer test strip (297 pg/mL) and the antibody test strip (3310 pg/mL), respectively. This significant increase in sensitivity likely can be attributed to a higher amount of detection ligands (i.e., sAptamers) printed on the T-line without causing unwanted false positive side effects, all of which are enabled by the streptavidin-G6.5-sAptamers printed on the T-line.

A closer detection performance analysis between streptavidin-G6.5-sAptamers and streptavidin-G3.5-sAptamers test strips showed significant differences. Although the number of the printed sAptamers on the T-line was estimated to be similar for both types of test strips, the streptavidin-G6.5-sAptamers test strips demonstrated a much more enhanced detection performance in that the limit of detection (LOD) was 188 times lower than the streptavidin-G3.5-sAptamers test strips (i.e., 24 vs. 4501 μg/mL). This observation is likely related to the combined effects of relative molecular sizes and steric hindrance. Studies have shown that the molecular sizes of streptavidin molecules are larger than that of the G3.5 molecules (molecular size diameter of 6.5 vs. 5.2 nm), and therefore when conjugated to the G3.5-sAptamer complexes, the streptavidin molecules would likely shadow sAptamers to prevent the sAptamers from effectively capturing their targets, such as the DENV S1 E protein on the T-line, thereby affecting detection performances.

The response curve of the streptavidin-G6.5-sAptamers test strips had a slope (k=0.038) in the linear region that was significantly greater than those of the streptavidin-sAptamer (k=0.035), antibody (k=0.020), and streptavidin-G3.5-sAptamers (i.e., streptavidin-G3.5-sAptamer) (k=0.020) test strips (p<0.05), strongly suggesting that the streptavidin-G6.5-sAptamers test strips have an evident advantage in enhancing detection sensitivities in the paper test strip detection platform.

To further investigate the reasons for such enhanced detection sensitivities afforded by the streptavidin-G6.5-sAptamers test strips, a second T-line was printed between the standard locations of the T-line and C-line (dual T-line format). As shown in FIG. 7B, streptavidin-sAptamer test strips clearly exhibited two positive T-lines regardless of the analyte concentrations used, strongly arguing that not all signaling molecules were effectively captured by the first T-lines and only to be captured by the second T-line. It is likely because that there were still signaling molecules that were not captured by the second T-line. The signaling molecule losses on the T-line is most likely the reason for the low detection sensitivities seen in most, if not all, standard test strips. In contrast, the streptavidin-G6.5-sAptamers test strip only showed the first positive T-lines and not the second positive T-lines in all analyte concentrations studied, indicating that there were minimal signal losses/leaks, likely due to significantly enhanced capturing of signaling molecules on the first T-lines. The results have clearly demonstrated the improved target capturing performance and significant advantages of the streptavidin-G6.5-sAptamers in enhancing the detection sensitivities of test strips.

### 4. Matrix Effect and Detection Specificity

To test the performance of the streptavidin-G6.5-sAptamers test strip in a simulated real-world environment, DENV S1 E protein samples of different concentrations (i.e., 3000, 30, 0.3 ng/mL) were prepared separately in PBS (pH 7.4) and BSA (1% w/w) solutions and used individually in strip tests. As shown in FIG. 8A, the signal intensities did not show significant differences for a given analyte concentration regardless of the sample matrix used, BSA or PBS (pH 7.4) (p>0.05). This result indicates that the streptavidin-G6.5-sAptamer LFA can reliably detect target samples in complex biological matrices.

To evaluate the detection specificity of the streptavidin-G6.5-sAptamers test strip for DENV S1 E protein samples, other non-target proteins, including DENV S2, S3, and S4 E proteins and Zika virus (ZIKV) E protein, were tested in parallel. As shown in FIG. 8B, the signal for the target protein (DENV S1 E protein) was significantly higher than that for non-target proteins (DENV S2, S3, and S4 E proteins and ZIKV E protein), indicating that the streptavidin-G6.5-sAptamer LFA has high specificity for detecting DENV S1 E protein.

As shown in Table 1, the LOD achieved in the current embodiment is much better than most reported in the literature using different dengue virus detection platforms. In addition, the advantages afforded by the strip test platform, including no requirement for special equipment and rapid sample-to-result turn-around in less than 15 minutes, strongly suggest that the current strip test platform has real potential to be a promising alternative to many existing detection platforms.

TABLE 1
Comparison of different methods for DENV detection

| | | | Detection | Detection | | |
|---|---|---|---|---|---|---|
| Targets | LOD | | Method | Time | | References |
| DENV2 E Protein | 0.5 | μg/mL | QCM | 1 | h | Ref. [3] |
| DENV1 NS1 | 0.03125 | ng/mL | ELISA | 2-3 | days | Ref. [4] |
| DENV NS1 | 0.3 | ng/mL | Electrochemical | 2 | days | Ref. [5] |
| | | | aptamer sensor | | | |
| DENV S1 | 0.024 | ng/mL | Dendrimer-aptamer | 15 | mins | This |

| | | |
|---|---|---|
| E Protein | LFA | Embodiment |

Note:
QCM is quartz crystal microbalance; ELISA is enzyme-linked immunosorbent assay; NS1is Dengue virus nonstructural protein 1.

### REFERENCES

1. Qin, Y., et al., Developing a non-fouling hybrid microfluidic device for applications in circulating tumour cell detections. Colloids and Surfaces B: Biointerfaces, 2017. 151: p. 39-46;
2. Barbara, K. and B. Maria, Review Dendrimers: properties and applications. Acta biochimica polonica, 2001. 48(1): p. 199-208;
3. Su, C.-C., et al., Development of immunochips for the detection of dengue viral antigens. Analytica chimica acta, 2003. 479(2): p. 117-123;
4. Ganguly, A., et al., Development of an ultrasensitive hetero-sandwich ELISA assay based on bispecific monoclonal antibody for the detection of dengue NS1 protein. journal of pharmacy research, 2013. 7(5): p. 374-380;
5. Rashid, S., et al., Label free ultrasensitive detection of NS1 based on electrochemical aptasensor using polyethyleneimine aggregated AuNPs. Microchemical Journal, 2020. 158: p. 105285.

While the invention has been described with reference to specific embodiments, the invention is not limited thereto, and various equivalent modifications and substitutions can be easily made by those skilled in the art within the technical scope of the invention. Therefore, the protection scope of the present invention shall be subject to the protection scope of the claims.

1. A bio-ink and its preparation method that include: a) coupling dendritic polymers with capturing ligands to form multi-handled polymer-ligand conjugate complexes, and b) coupling the multi-handled polymer-ligand conjugate complexes with specific proteins to obtain the bio-ink.

2. The method to prepare the bio-ink, according to claim 1, is characterized in that the dendritic polymer is a poly(amidoamine) PAMAM dendrimer, poly(propylenimine), triazine, phosphorus, or polyether dendritic polymer.

3. The method to prepare the bio-ink, according to claim 2, is characterized in that the PAMAM dendrimer includes dendrimers from generation 2 to generation 9.

4. The method to prepare the bio-ink, according to claim 2, is characterized in that the capturing ligand includes aptamers, antibodies, peptides, and molecularly imprinted polymers.

5. The method to prepare the bio-ink, according to claim 4, is characterized in that the sequence of the aptamer is shown as in SEQ ID NO. 1.

6. The method to prepare the bio-ink, according to any of the above claims from 1 to 5, is characterized in that the specific protein includes streptavidin, avidin, and bovine serum albumin.

7. The bio-ink prepared by the method according to any of the above claims from 1 to 6.

8. The application of the bio-ink was prepared by the method according to claim 7 in a strip test kit.

9. A dengue virus strip test kit, in a lateral flow test strip format that includes a sample pad, a conjugate pad, and a membrane with a printed test line and control line, is characterized in that the test line contains bio-ink for detecting dengue virus, wherein the preparation of the bio-ink includes: a) coupling PAMAM dendrimers with capturing ligands to form conjugate complexes, wherein the capturing ligands are amine-functionalized aptamers with the sequence as shown in SEQ ID NO. 1; and b) coupling the conjugate complex with streptavidin to obtain the bio-ink.

10. The dengue virus test kit according to claim 9, is characterized in that the conjugate pad is labeled with oligonucleotides-AuNPs, wherein the sequence of the oligonucleotides is shown as in either SEQ ID NO. 2 or SEQ ID NO. 3; the control line contains a biotin-streptavidin complex, wherein the sequence of biotin is shown as in SEQ ID NO. 4.


SEQUENCE LISTING (4 sequences)

SEQ ID NO: 1 - DNA, 72 bp, synthetic construct
  source 1..72: mol_type = other DNA
atccgtcacacctgctctaggctgtggtgacgtaccaggggagtgggtcgccagtggtgttggctcccgtat

SEQ ID NO: 2 - DNA, 69 bp, synthetic construct
  source 1..69: mol_type = other DNA
atccgtcacacctgctctggtagttcgttctcttgtacactctggaatttagggtggtgttggctcccg

SEQ ID NO: 3 - DNA, 20 bp, synthetic construct
  source 1..20: mol_type = other DNA
aaaaaaaaaaaaaaaaaaaa

SEQ ID NO: 4 - DNA, 20 bp, synthetic construct
  source 1..20: mol_type = other DNA
tttttttttttttttttttt